(19) United States
(12) Patent Application Publication
(10) Pub. No.: US 2006/0207329 A1
(43) Pub. Date: Sep. 21, 2006

(54) Ultrasonic evaluation of the strength of flour doughs

(76) Inventors: John H Page, Manitoba (CA); Husein M Elmchdi, Manitoba (CA); Martin G Seanlon, Manitoba (CA)

Correspondence Address: Michael R Williams;Ade & Company, 1700-360 Main Street, Winniepg, MB, R3C 3Z3, CA

(21) Appl. No.: 10/525,411

(22) PCT Filed: Aug. 21, 2003
(86) PCT No.: PCT/CA03/01249

§ 371 Date: Mar. 17, 2006

Related U.S. Application Data
(60) Provisional application No. 60/404,781, filed on Aug. 21, 2002.

(51) Int. Cl. G01N 33/10; G01N 29/024; G01N 29/032; G01N 29/07; G01N 29/11; G01N 3/08

(52) U.S. Cl. 73/597; 73/599; 73/169; 73/822

(57) ABSTRACT

A method of determining dough strength and predicting loaf quality is herein described. The method involves propagating an ultrasound signal through a sample of dough and determining the transit time and amplitude of the ultrasound signal, and hence the ultrasonic velocity and attenuation. These data are then used to determine dough strength and predict product quality. Measurement of dough expansion by digital photography as pressure is varied provides complementary information that is used to determine dough strength and predict product quality

This application claims priority under 35 USC § 119(e) to Provisional Patent Application Ser. No. 60/404,781 filed on Aug. 21, 2002.

### FIELD OF THE INVENTION

The present invention relates generally to the field of food quality. More specifically, this invention relates to a method of evaluating the strength of flour doughs.

### BACKGROUND OF THE INVENTION

Bread, in its simplest composition, is a baked mixture of finely ground cereal flour, salt and water. Baking causes the partial gelatinization of the starch and that permits the bread to be digestible. Bread has a palatable texture due to its aerated structure. The aerated structure of bread is made possible by the ability of the gluten proteins, which are found almost uniquely in wheat flour, to form a gas-trapping network when mixed with water and developed into a dough. With the addition of yeast, which metabolizes sugars to produce carbon dioxide, this gas-trapping ability allows production of the aerated dough which is set by baking to give raised bread.

When the loaf of bread is taken out of the oven, it consists of crust and breadcrumb. The main difference between the crust and the breadcrumb is the difference in the temperature they attain during baking. The high temperature of the crust causes the evaporation of water so that the water content of the crust is low compared to that of the breadcrumb. From the structural point of view, the breadcrumb is a pore structure consisting of the gas cells and pore walls, called the matrix (Zghal et al., 1999; Zghal, 1999). The pore walls consist of the partly gelatinized starch, and a monolayer lipid film with patches of polymerized high molecular weight storage protein units dispersed within it (Eliasson and Larsson, 1993). The crust on the other hand, is a hard, vitreous surface layer formed of collapsed crumb pore walls. It is a continuum of dried starch gel with protein and lipid aggregates (Eliasson and Larsson, 1993).

Dough rheology focuses on the viscoelastic properties of bread dough, for example, the rate at which the internal stress induced by mechanical treatment relaxes during the rest period and which depends on both on the viscosity and the elasticity of the dough (Matsumoto and Nishiyama, 1973, Matsumoto et al., 1971). The methods used to study dough rheology have relied on instruments such as dough recording mixers like the farinograph, which provides information about the behavior of the dough during the mixing stage; load-extension instruments like the Extensigraph (deformation in one direction) and Alveograph (deformation in two directions) which yield information on the dough's resistance to extension, which is then related to gas retention or gas holding capacity during fermentation. Even though these instruments and methods investigate the properties of the dough as a whole, they fail to depict the true contribution of the air bubbles to dough rheology. The air bubbles are present in considerable number and in the final product, the loaf, they represent much more than half of the total volume. Knowledge is required of the effect of air bubbles on the properties of the dough during mixing and during proofing, so as to gain an understanding of the effect of the gas cells on the mechanical properties of breadcrumb, and on the resulting quality of the loaf of bread made from the dough.

Although ultrasonic techniques are commonly used in materials science for investigating the mechanical properties of inorganic materials, their application to biological systems is less well established. Ultrasonic velocity measurements can be used to determine the stiffness or rigidity of the material, as can be most clearly seen by expressing the velocity in terms of the (dynamic) elastic modulus. Ultrasonic attenuation is especially sensitive to the structure of inhomogeneous materials at a resolution determined by the wavelength.

Despite its potential, the use of ultrasound in investigating the properties of bread dough and breadcrumb is very limited. Moorjani (1984) was the first to use ultrasound to investigate the properties of bread dough (with and without yeast). She measured the ultrasonic velocity of dough and fresh breadcrumb using 25 kHz transducers. Her measurements were limited to ultrasonic velocity. In 1992, Lee et al., used ultrasonic shear measurements as a technique to investigate the rheological properties of bread dough. They demonstrated that rheological properties of bread dough can be measured non-destructively at ultrasonic frequencies ranging between 0.3 and 1 MHz. The third research group is that of Letang et al. (1996), who investigated the acoustic properties of water-flour mixtures as a function of water content. Their range of frequencies was between 2 MHz and 8 MHz, which corresponds to very short wavelengths. None of these research groups have investigated the elastic properties of the dough with a focus on the presence of the air bubbles or gas cells. The significance of gas cells in determining the quality of bread products has been highlighted by Campbell (1991) and Campbell et al. (1998), who articulated the view that breadmaking is essentially a series of aeration processes that must be understood and optimized to produce a well aerated loaf with an attractive volume and desirable textural attributes. The bubbles are incorporated into the dough during mixing and act as nucleation sites for carbon dioxide produced by the yeast metabolism during proofing. The development of the dough structure allows these bubbles to be inflated with carbon dioxide without excessive coalescence or loss of gas.

### SUMMARY OF THE INVENTION

According to a first aspect of the invention, there is provided a method of determining dough quality comprising:

a) inserting a quantity of dough into a receptacle;

b) propagating an ultrasound signal through the dough;

c) determining ultrasonic velocity and attenuation of the ultrasound signal after passing through the dough; and

d) predicting dough quality based on the ultrasonic velocity and attenuation of the ultrasound signal.

According to a second aspect of the invention, there is provided a method of determining dough quality comprising:

a) inserting a quantity of dough into a receptacle;

b) propagating an ultrasound signal through the dough at a first temperature;

c) determining ultrasonic velocity and attenuation of the ultrasound signal after passing through the dough;

d) repeating steps (a) through (c) at at least one other temperature; and

e) predicting dough quality based on the ultrasonic velocity and attenuation of the ultrasound signal versus temperature.

According to a third aspect of the invention, there is provided a method of determining dough quality comprising:

a) inserting a first quantity of dough into a first receptacle, said first receptacle having a first thickness;

b) propagating an ultrasound signal through the first dough;

c) determining of the ultrasound signal after passing through the first dough;

d) inserting a second quantity of dough into a second receptacle, said second receptacle having a second thickness;

e) propagating an ultrasound signal through the second dough;

f) determining transit time and amplitude of the ultrasound signal after passing through the second dough; and

g) predicting dough quality based on the ultrasonic velocity and attenuation of the ultrasound signal from the thickness dependence of the transit time and amplitude.

According to a fourth aspect of the invention, there is provided a method of analyzing fermentation response in a quantity of dough comprising:

(a) inserting a quantity of dough into a receptacle having a given thickness;

(b) propagating an ultrasound signal through the dough at a first time;

(c) determining ultrasonic velocity and attenuation of the ultrasound signal after passing through the dough at said first time;

(d) propagating an ultrasound signal through the dough at a second time;

(e) determining ultrasonic velocity and attenuation of the ultrasound signal after passing through the dough at said second time; and

(f) determining fermentation response of the dough based on the change in ultrasonic velocity and attenuation over time.

According to a fifth aspect of the invention, there is provided a method of determining dough quality comprising:

(a) inserting a quantity of dough into a receptacle having a given thickness, said receptacle being in a chamber in which pressure can be varied;

(b) propagating an ultrasound signal through the dough at a first pressure;

(c) determining ultrasonic velocity and attenuation of the ultrasound signal after passing through the dough at said first pressure;

(d) propagating an ultrasound signal through the dough at a second pressure;

(e) determining ultrasonic velocity and attenuation of the ultrasound signal after passing through the dough at said second pressure; and

(f) predicting dough quality based on the change in ultrasonic velocity and attenuation versus pressure.

According to a sixth aspect of the invention, there is provided a method of determining dough quality comprising:

(a) inserting a quantity of dough into a receptacle having a given thickness, said receptacle being in a chamber in which pressure can be varied;

(b) determining the area of dough expansion within the receptacle by digital photography at said first pressure;

(c) determining the area of dough expansion within the receptacle by digital photography at a second pressure;

(d) predicting dough quality based on the change in true strain of dough sample versus pressure.

### BRIEF DESCRIPTION OF THE DRAWINGS

FIG. 1 is a block diagram describing the experimental set-ups: (A) for propagation of ultrasonic signal through the dough; (B) for determination of dough expansion.

FIG. 2 shows a typical set of results for ultrasonic signal amplitude as a function of sample thickness for different mixing pressures.

FIG. 3 shows the attenuation coefficient of dough as a function of void fraction.

FIG. 4 shows the transit time through the sample as a function of sample thickness for samples taken from the same dough piece.

FIG. 5 shows the velocity of sound through dough mixed at various pressures. The solid line represents Wood's prediction.

FIG. 6 shows the velocity of ultrasound in fermenting dough mixed under vacuum and at atmospheric pressure.

FIG. 7 shows the change in the attenuation coefficient for fermenting dough mixed under vacuum and at atmospheric pressure.

FIG. 8 is a plot of attenuation coefficient versus mixing time versus for flour dough made from the wheat cultivar AC Reed.

FIG. 9 is a plot of ultrasonic velocity versus mixing time for flour dough made from the wheat cultivar AC Reed.

FIG. 10 is a plot of attenuation coefficient versus mixing time versus for flour dough made from the wheat cultivar Corinne.

FIG. 11 is a plot of ultrasonic velocity versus mixing time for flour dough made from the wheat cultivar Corinne.

FIG. 12 is a plot of ultrasonic velocity (A) and signal amplitude (B) versus pressure in the ultrasonic alveograph for flour dough made from CWRS wheat flour.

FIG. 13 is a plot of ultrasonic velocity versus pressure in the ultrasonic alveograph for flour dough made from CWRS wheat flour demonstrating the hysteresis that occurs in velocity when pressure is reduced and then raised.

FIG. 14 is a plot of stress versus true strain obtained from the imaging embodiment of the ultrasonic alveograph for flour dough made from three classes of wheat at a moisture content of 63 ml of water per 100 g of flour with three replicates for each wheat class, demonstrating the excellent reproducibility of the measurements.

FIG. 15 is a plot of stress versus true strain obtained from the imaging embodiment of the ultrasonic alveograph for flour dough made from two classes of wheat at a moisture content of 52 ml of water per 100 g of flour showing the differences in rheology between the doughs made from the two wheat classes.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein is a device for and a method of evaluating the strength, rheological properties, gas entrainment capacity and fermentation response of flour doughs. As discussed below, a quantity of dough is prepared and mixed using means known in the art. A sample of the dough is then removed and prepared for analysis by the device. Specifically, the dough sample is placed into a holder arranged to accept the dough sample therein and low intensity ultrasound is propagated from an emitter through the sample into a receiver. In some embodiments, the holder is fitted between an ultrasonic emitter and an ultrasonic receiver, for example, at least two ultrasonic transducers. Transit time and amplitude of the ultrasonic signal at this one thickness are then used to predict end-product quality. In some embodiments, the thickness of at least one other dough sub-sample is varied and the process is repeated. Plots of transit time and amplitude of ultrasonic signal versus sample thickness are then created. As discussed below, these are used to calculate the ultrasonic velocity and attenuation, for example, by plotting log(amplitude) against distance, which are then used to predict end-product quality, for example, loaf quality. It is of note that these parameters may be calculated by other means which are within the scope of the invention. In other embodiments of the invention, the temperature at which the ultrasonic measurements are obtained is varied to provide additional information on dough quality. In these and other embodiments, a second dough sample which varies from a first in at least one variable affecting gas entrainment is then analyzed as described above. For example, the variable affecting gas entrainment may be mixing time or headspace pressure during mixing. The additional information on the change in dough properties measured by ultrasound as a function of gas entrainment permits further assessments of end-product quality.

As will be appreciated by one of skill in the art, one manner for varying the thickness of the dough sample is to use two or more receptacles having substantially similar properties when used within the invention but having cavities or openings of differing thicknesses. Thus, when the respective dough samples are inserted into these receptacles, the respective dough samples will have different thicknesses. It is of note that as used herein, “thickness” when referring to a receptacle, refers to the thickness of a dough sample when inserted into the receptacle.

As will be appreciated by one of skill in the art, predicting loaf quality during the mixing stage represents a significant improvement over current methods wherein loaf quality is not evaluated until after baking. Specifically, using the instant method, it is possible to predict loaf quality at the dough mixing stage and if necessary take corrective appropriate steps or discard unsuitable dough, thereby saving time, expense and resources. As will be apparent to one of skill in the art, “appropriate steps” may include, for example, but by no means be limited to, addition of matrix improvers, surface-active agents and oxidants. More specifically, by way of examples, if the ultrasound results indicate that the dough possesses inadequate strength, matrix improvers, such as glucose oxidase, may be added to subsequent mixes to increase dough strength. The stronger more elastic dough obtained by this corrective action exhibits greater dough stability with greater resistance to mechanical handling in subsequent dough processing operations, thus improving the crumb structure and the loaf volume of the end-product as a result of utilization of dough strength information provided by the invention. As will be apparent to one of skill in the art, the selection of a suitable surface-active agent from the many various trade names for modification of the surface tension between matrix and gas cell phases will depend on the dough manufacturers knowledge of their product and the degree of modification of dough properties that they wish to effect based on their interpretation of the ultrasound results. It is also important to note that as discussed below suitable or acceptable transit times and amplitudes will vary according to preferences and end products.

According to one embodiment of the invention, there is provided a method of determining the strength of a flour dough comprising: providing a quantity of a flour dough, subjecting the dough to ultrasonic energy, recovering and analyzing the ultrasonic data, thereby determining dough strength.

According to another embodiment of the invention, there is provided a method of analyzing fermentation response in dough wherein the ultrasonic data are analyzed over time and the change in transit time and attenuation is a measure of the fermentation response. As will be appreciated by one of skill in the art and as discussed herein, attenuation measures the gas entrainment of the dough, and in this instance, the change in attenuation measures incorporation of CO₂ into the gas cells during fermentation.

In one embodiment, the dough is mixed at a single pressure and a single mixing time. A first sample is then inserted into the sample holder such that the sample has a given thickness and low intensity ultrasound is then propagated therethrough. The transit time and amplitude of the ultrasonic signal is recorded. The process is repeated for a second sample having a different thickness. From these data, transit time and amplitude (log(amplitude)) are plotted against sample thickness which are used to determine ultrasonic velocity and attenuation and in turn predict end product quality.

As will be appreciated by one of skill in the art, two points are the minimum needed for determining the slope of a line; however, data from more than two thicknesses would of course provide a more accurate result.

As discussed herein, it was previously believed that it was necessary to analyze samples of dough mixed at different pressures in order to predict loaf quality using the instant invention. However, it has since been discovered that as discussed below, different pressures are not the only means to entrain different quantities of air, as the use of differing mixing times is also sufficient. As will be apparent to one of skill in the art, this represents a significant improvement, as the time and effort needed to prepare samples at multiple pressures is not required. Furthermore, it has been noted that the developed methods and formulas may also predict loaf quality at a single pressure and single mixing time.

It is also of note that the invention as described herein may be used to predict the quality of other products made from flour doughs. It is further of note that in these and other embodiments, acceptable values may be above or below a threshold value, or may fall within an acceptable range. This will of course depend upon the specific product being made from the dough.

In some embodiments, dough is mixed for multiple, for example, at least two, mixing times. As will be appreciated by one of skill in the art, mixing is solely responsible for generating the nuclei that develop into gas cells within the bread crumb, and is critical for achieving the optimal development of the protein network that is vital for maximum retention of gas generated by the leavening agents in subsequent processing operations. Thus, dough mixed for different time periods will entrain different quantities of air. In the instant invention, ultrasonic attenuation directly probes the amount of entrained gas while the ultrasonic velocity varies dramatically with the rigidity of the dough matrix, allowing effects arising from chemical changes in the matrix to be measured.

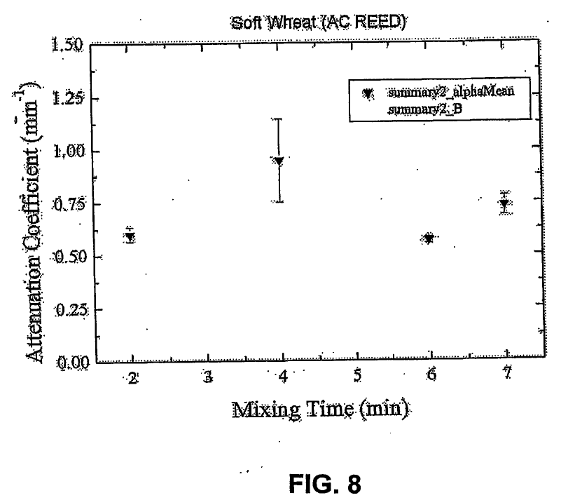

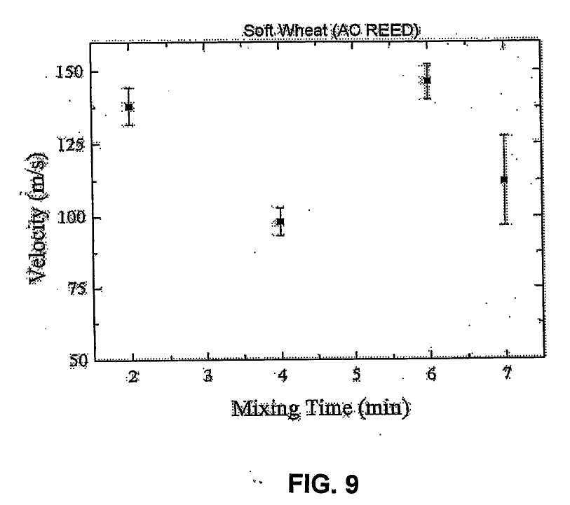

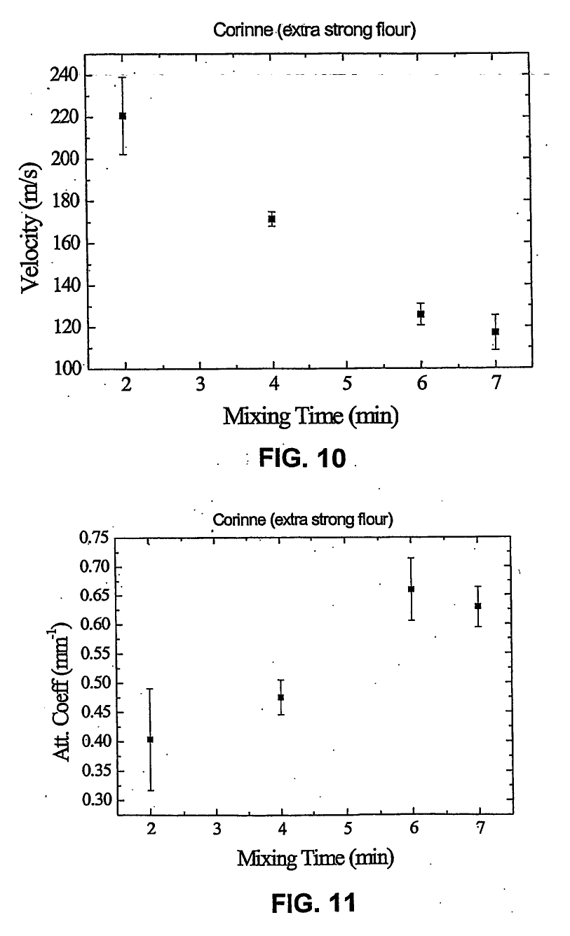

As will be appreciated by one of skill in the art, flour varies considerably in how effectively its proteins respond to the work input from the mixer to form a cohesive viscoelastic matrix and how readily the flour can entrain air per unit time of mixing. Specifically, the faster uptake of air by doughs made from varieties of poorer breadmaking quality will be measured by ultrasound, so that a characterization of the change in ultrasonic velocity and attenuation as a function of mixing time will permit the user to evaluate the strength of flour doughs of unknown origin or strength. This information can be exploited by allowing bakers to optimize both the matrix and gas cell properties of the dough during mixing through the addition of matrix improvers, surface-active agents and oxidants, based on knowledge of the strength of the flour. Examples of data from a strong flour are shown in FIGS. 10 and 11 while examples of data from a weak flour are shown in FIGS. 8 and 9.

As will be appreciated by one of skill in the art, the evaluation of the breadmaking quality of a flour is extremely complicated (Bushuk, 1975). To avoid performing the protracted and experimentally difficult baking evaluations, cereal scientists have developed various simple methods for evaluating dough strength to attempt to predict the breadmaking quality of a flour. Optimum dough strength is usually defined as the propensity of the wheat flour to make bread of desirable quality. This varies according to the product and the process being used to create the bread, but usually encompasses traits of adequate loaf volume with satisfactory crust and crumb characteristics. During mixing, doughs made from weak flours develop rapidly, break down quickly, and are unable to tolerate variation in mixing time. Doughs made from strong flours may have good mixing stability, but bakers may have difficulty in establishing optimal processing conditions for doughs made from such strong flours. One problem for the baker is knowing how to reliably and accurately establish acceptable or desirable mixing properties from such dough strength evaluations (Tipples, 1975), in order that a desired amount of gas is entrained during mixing, and/or that appropriate revisions to ingredients can be made to alter the properties of the matrix. In this manner, the baker can make better use of a given flour to consistently produce a product of acceptable end-quality. The use of ultrasonic evaluation of these aggregate properties as defined by dough strength will provide the baker with accurate and precise information on dough properties pertinent to the baker's processing conditions and desired product quality attributes. In addition, the ability of ultrasonic attenuation to measure gas entrainment in the dough means that this invention has a distinct advantage for comprehensively determining the breadmaking potential of wheat flours.

As discussed above, the device comprises a sample holder, an ultrasonic emitter, an ultrasonic receiver and a compiler arranged to analyze the ultrasonic data.

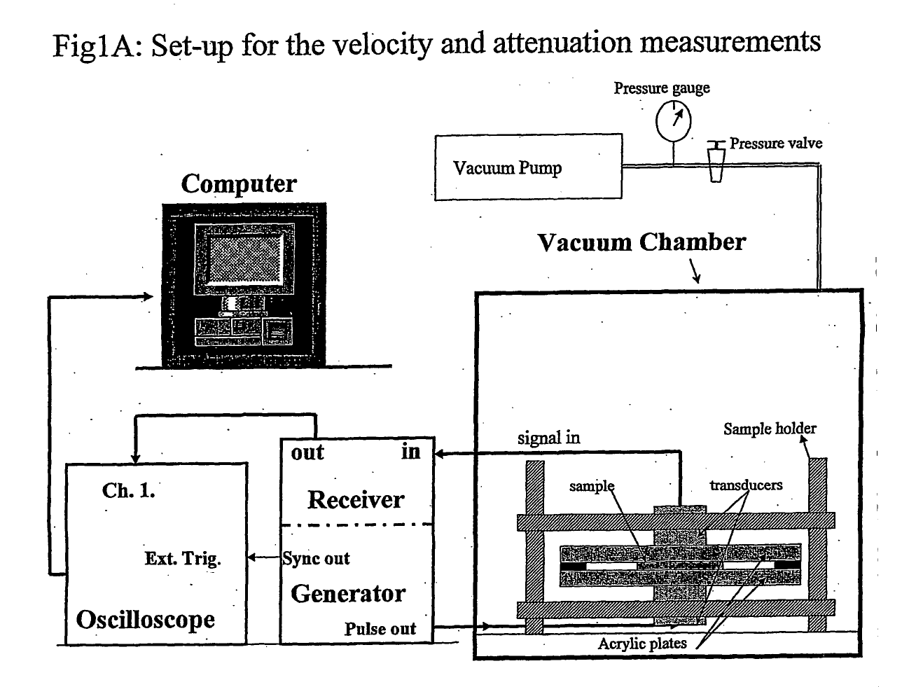

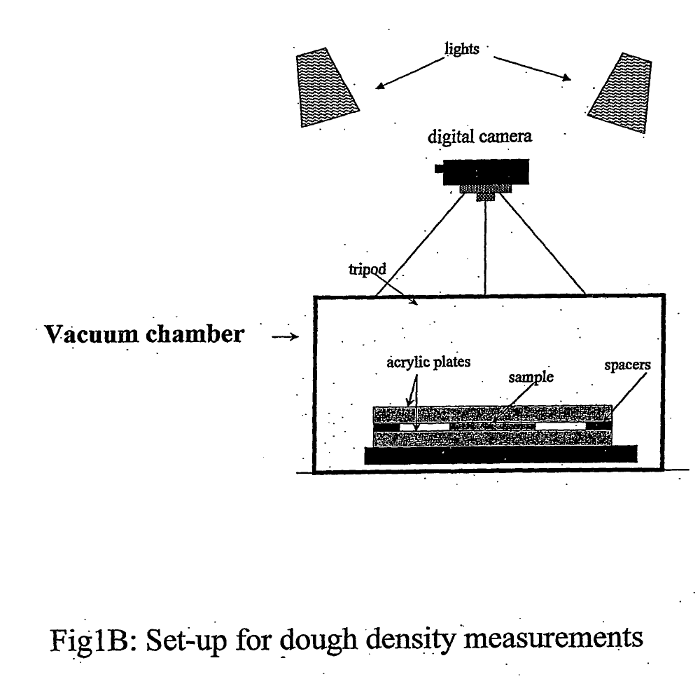

The ultrasonic emitter and receiver were mounted in a custom-made holder. The purpose of the holder was to support the emitter and receiver so that the gap between them was maintained constant, thus accurately controlling the sample thickness. The holder also served to control the parallel alignment of the emitter and receiver (e.g., ultrasonic transducers), so that the top and bottom surfaces of the sample were parallel. In one embodiment, the emitter and receiver were supported by two aluminum plates, as shown in FIG. 1A, thereby defining a cavity of predetermined thickness to accept the sample. In yet other embodiments, other suitable sample holders may also be used. Specifically, it is of note that a suitable holder will be arranged to accept a dough sample, having a cavity or gap of accurate dimensions. In yet other embodiments, flat acrylic plates may be fitted between the ultrasonic emitter and receiver, so that the dough is confined between smooth uniform surfaces thereby allowing the dough to expand reproducibly so that the dough's fermentation response can be monitored.

It is of note that no new gas cells can be generated after the mixing stage. Therefore, entrainment of the right amount of gas and its “proper” distribution throughout the dough during mixing is vital to the final quality of the breadcrumb. Some bakeries employ mixing under different pressures—elevated initially to get oxygen in to modify the properties of components within the cell walls so that the matrix is strong, and under vacuum in the latter stages of mixing to get a small volume of gas in the dough to ensure that a good crumb structure develops. In addition to mixer headspace pressure, the “proper” distribution of gas cells in the dough depends on the viscosity of the dough, the concentration and type of surfactants, and the type of mixer.

As will be apparent to one of skill in the art, fermentation response refers to the fact that as time progresses the metabolic activities of the yeast generate CO₂ which expand the gas cells which have been formed during mixing. If the dough is of poor quality or has been improperly mixed then this gas is either not well retained (so that there is a loss in loaf volume) or the dough is not ‘strong’ enough to prevent adjacent gas cells from coalescing. The resulting crumb structure is then poor due to large holes in the crumb where wholesale coalescence of gas cells has occurred.

As will be appreciated by one of skill in the art, dough has a high attenuation. In some embodiments, the ultrasound frequency used in the instant invention is preferably a frequency between 18-100 kHz, or more preferably, 40-75 kHz. It is further of note that in some embodiments, audible frequencies, that is, frequencies of less than 18 kHz may be utilized. This range of frequencies is important to the success of the invention, since frequencies in this range are needed for ultrasound to be a sensitive probe of the gas bubbles in dough and their interaction with the dough matrix. At higher frequencies in the kilohertz range, the attenuation of longitudinal ultrasound becomes very large due to resonant scattering and absorption by the gas bubbles, making measurements difficult if not impossible. At still higher frequencies in the megahertz range, the gas bubbles have little effect on the velocity and attenuation of ultrasound, and the physical mechanism underlying the invention is no longer operative.

The great advantage of ultrasound compared with other non-intrusive methods such as light scattering is that the majority of food materials do transmit ultrasound even though they may be optically opaque. Therefore, there remains a wide range of liquid and solid foods which are amenable to analysis using ultrasonic methods.

In general, the propagation of ultrasound through a system depends upon its response to rapid pressure fluctuations. Foods are rarely homogeneous, and the transmission of ultrasound through a multi-phase material such as dough is influenced not only by the properties of the various phases in isolation, but also by the physical structure. These structural features include the concentration, size and distribution of phases or particles, and ultrasound sensitivity to these features depends on the mismatch in the acoustic properties of the constituents.

The features of ultrasonic wave propagation of which most use has been made experimentally are the velocity and attenuation of the wave.

When an ultrasonic wave propagates through a heterogeneous system, the wave may be scattered as well as absorbed. Scattering occurs whenever the constituent materials have different densities and/or phase velocities. Relatively simple examples of such heterogeneous systems consist of spherical particles, voids or inclusions distributed throughout a second material, with the second medium forming a continuous phase. In dough, the gluten matrix encompasses a high volume fraction of another phase—the starch granules. A third phase is also contained within the gluten matrix, but initially at a lower volume fraction: the gas bubbles. The largest difference in both density and velocity is between the gas bubbles and the matrix, so that the strongest scattering is from the gas bubbles. The magnitude of the scattering at a given frequency is strongly dependent on the size of the dispersed bubbles; thus the scattering can be either enhanced or reduced by suitable choice of the ultrasonic frequency. However, one crucial point here is that the presence of scattering can produce a distortion of the wavefront when a plane wave propagates through the material. This occurs because, in addition to the ballistic component that travels straight through the material without scattering out of the forward direction, there is also a scattered component, which in general varies in both amplitude and phase across the output face of the sample. For dough, absorption dominates over scattering. Since the ballistic signal propagates according to:
A=A₀exp(−αx/2)exp{i(kx−ωt)},
where A₀ is the amplitude at x=0, α is the attenuation coefficient in m⁻¹, k=2π/λ is the wave number, λ is the wavelength and ω is the angular frequency (=2πf),

its measurement allows quantitative information to be obtained on how both absorption and scattering influence the attenuation and phase velocity.

The structural properties of dough were investigated by studying the behavior of the longitudinal ultrasonic signal as it propagates through the samples. The ultrasonic parameters that characterize the propagation of the ultrasonic signal are the phase velocity and attenuation coefficient. The numerical values of these two parameters change as the structure of the material is altered, thereby providing a tool for monitoring these internal changes in the structure of the material. For example, in the simple case of voids in a homogeneous matrix, attenuation and velocity will change as a function of the volume fraction of voids.

In other embodiments of the invention, temperature is a controlled variable given that:
a) material parameters in viscoelastic materials (such as dough) are a function of temperature, so that variability in velocity and attenuation is expected if temperature is uncontrolled.
b) leavening, either biochemically by yeast, or chemically by ingredients, is very sensitive to temperature. Therefore, to control the rate at which carbon dioxide is generated, and thus determine the effect of leavening agents on the measured ultrasonic parameters of the dough, temperature must be controlled.

Alternatively, it is possible to induce changes in the properties of the dough by altering temperature, and monitoring how the ultrasonic parameters vary as a function of temperature. This is particularly appropriate if the temperature of the dough is raised sufficiently to pass through two important thermal transitions in the dough—the denaturation of the gluten proteins within the dough (circa 50 C), and the gelatinization of the dough's starch (60-75 C). The nature of these transitions in doughs made from flours of different dough strength are reasoned to affect breadmaking potential. By continuously monitoring ultrasonic parameters as the dough passes through these transitions, information can be obtained from plots of (for example) velocity versus temperature (both during heating and cooling) that can then be related to the quality potential of the doughs to be manufactured into cereal products such as, but not limited to, doughs, cookies and noodles.

In one embodiment of the invention, there is provided an ultrasonic alveograph. In a conventional alveograph, a dough is made into a sheet and air is blown into the sheet to generate a bubble, which is then inflated until it ruptures. The pressure in the dough bubble and the time are recorded so that from the “alveogram” certain parameters are obtained that characterize the rheology of the dough. It is claimed that the biaxial extension of the dough sheet generates deformation in the dough that is more similar to the deformation that occurs in a real dough piece that is subjected to fermentation and oven rise. Thus, in assessing the potential baking performance of a wheat flour dough, greater applicability of this dough rheology test is claimed over other quality evaluation techniques based on measurement of dough rheology. In the embodiments of this invention, a dough sample (made without yeast) is placed in a chamber in which a vacuum can be drawn. The transit time and amplitude of an ultrasonic pulse propagating through the dough is measured continuously (or at specific time intervals) as a vacuum is drawn to reduce pressure in the chamber to a specific end-value of pressure. The rate at which the vacuum is drawn can be matched to the time interval, so that readings of transit time and amplitude are made at known pressures in chamber.

This procedure permits dough quality to be determined by measuring the changes in ultrasonic velocity and attenuation that are caused by the expansion of gas bubbles due to the reduction in the pressure surrounding the dough. Thus, the apparatus determines dough quality by a method that is similar to alveography, in which the properties of dough are measured as a function of biaxial extension of the dough. However the new proposed method is not subject to drawbacks of preparing a dough sheet and expanding it by an external air pressure source. In this technique, air bubbles naturally entrained in the dough during mixing are the source of the gas pressure, and so the triaxial expansion that occurs more accurately represents the gas cell expansion that occurs during fermentation. The advantages claimed for the biaxial deformation of alveography over quality evaluation from uniaxial deformation tests can be taken one dimension further in our technique in that we have triaxial extension of the dough by the expanding air bubbles.

When a specific end-value of pressure is attained, the vacuum is slowly released and the transit time and amplitude of the ultrasonic signal is measured as the pressure in the chamber rises.

The decrease in gas bubble size resulting from an increase in pressure above ambient may also be used to probe the dough properties, and the corresponding changes in velocity and attenuation monitored to predict dough quality.

In a separate embodiment, the change in volume V of the dough resulting from the change in external pressure is measured directly by placing the dough sample between two acrylic plates and recording the size of the dough slab using digital photography. The method therefore allows fundamental information on the bulk expansion of the dough to be obtained as the external pressure is reduced or increased. Since the stress in the dough is applied locally and in three dimensions, the method gives new information that complements conventional rheology measurements, in which the stress is applied at the surface of the sample. Thus fundamental new information on dough rheology can be obtained that is potentially more relevant to assessing baking quality. A second application of the method is to allow the rate of change of density or volume to be compared with the ultrasonic velocity and attenuation in the dough relaxation measurements described in the previous paragraphs.

Thus, the above-described process may be used for a variety of purposes:

1. Determine wheat flour dough quality of different cultivars in wheat breeding programs.

2. Determine crop quality in a given year and location for specific cultivars or classes of wheat to assist millers in raw material sourcing.

3. Assess effect of baking absorption on dough quality. Baking absorption might be manipulated by milling process, by, for example, altering starch damage. Baking absorption is a measure of how much water the dough can “carry”, and thus it relates to profitability in a bakery.

4. Assess tolerance of given cultivar or class of wheat to increases in flour extraction by the miller.

5. Assess effect of specific ingredients on improvement of dough quality in flours that have been prepared by any means in applications 1-4. Of particular note as bakery improving ingredients are oxidizing improvers, enzyme improvers and surfactants (emulsifiers).

6. Assess effect of specific processing modifications during dough mixing (e.g., work input, rate of work input, means by which shear energy is applied) on improvement of dough quality by flours prepared by any means in applications 1-5.

7. Assess how gases (of different composition to air) used in the headspace of the mixer during mixing interact with flour quality and/or ingredients to modify dough quality (particularly when the gases interact with oxidizing improvers such as, but not limited to, ascorbic acid).

8. Assess effect of kneading and moulding operations on the properties of dough.

9. Assess effect of any or more of applications 1 to 7 on fermentation response, and in particular provide quantitative assessment of the development of gas cells.

10. Assess effect of chemical leavening agents in application 9 by their substitution for yeast.

11. Assess effect of frozen storage on degradation of dough quality, and in particular, but not limited to, to determine whether extent of degradation is due to impairment of gluten quality or impairment of yeast or leavening agent activity.

12. Provide academic and industrial researchers with a comprehensive set of rheological tools in one invention that can be used to provide quantitative information on dough properties for a variety of analyses in cereal chemistry and dough processing studies.

In one embodiment of the invention, there is provided a method of determining dough quality comprising:

a) inserting a quantity of dough into a receptacle;

b) propagating an ultrasound signal through the dough;

c) determining ultrasonic velocity and attenuation of the ultrasound signal after passing through the dough; and

d) predicting dough quality based on the ultrasonic velocity and attenuation of the ultrasound signal.

The ultrasonic velocity and attenuation of the ultrasound signal may be determined by measuring transit time and amplitude of the ultrasound signal after passing through the dough. The ultrasound signal is a low-level ultrasound signal, for example, in the frequency range between 18-100 kHz.

It is of note that the transit time and the amplitude are determined relative to a reference pulse.

Steps (a)-(c) may be repeated for a second quantity of dough-differing from the first quantity of dough in at least one variable affecting gas entrainment, for example, mixing time, and headspace pressure during mixing.

In another embodiment, there is provided a method of determining dough quality comprising:

a) inserting a quantity of dough into a receptacle;

b) propagating an ultrasound signal through the dough at a first temperature;

c) determining ultrasonic velocity and attenuation of the ultrasound signal after passing through the dough;

d) repeating steps (a) through (c) at at least one other temperature; and

e) predicting dough quality based on the ultrasonic velocity and attenuation of the ultrasound signal versus temperature. The temperature may be varied for example from between 20° C. to 75° C., although other suitable temperature ranges may also be used.

In another embodiment of the invention, there is provided a method of determining dough quality comprising:

a) inserting a first quantity of dough into a first receptacle, said first receptacle having a first thickness;

b) propagating an ultrasound signal through the first dough;

c) determining transit time and amplitude of the ultrasound signal after passing through the first dough;

d) inserting a second quantity of dough into a second receptacle, said second receptacle having a second thickness;

e) propagating an ultrasound signal through the second dough;

f) determining transit time and amplitude of the ultrasound signal after passing through the second dough; and

g) predicting dough quality based on the ultrasonic velocity and attenuation of the ultrasound signal from the thickness dependence of the transit time and amplitude.

For doughs mixed at atmospheric pressure, the range of thicknesses used may be for example, from 0.5 mm to 3 mm. However, for dough mixed at reduced pressures, a range of 0.5 mm to 7 mm may be more appropriate, as the signal propagates better in the dough with lower gas content, all other factors being equal. It is of note that other suitable thicknesses may also be used and are within the scope of the invention.

According to another aspect of the invention, there is provided a method of analyzing fermentation response in a quantity of dough comprising:

(a) inserting a quantity of dough into a receptacle having a given thickness;

(b) propagating an ultrasound signal through the dough at a first time;

(c) determining ultrasonic velocity and attenuation of the ultrasound signal after passing through the dough at said first time;

(d) propagating an ultrasound signal through the dough at a second time;

(e) determining ultrasonic velocity and attenuation of the ultrasound signal after passing through the dough at said second time; and

(f) determining fermentation response of the dough based on the change in ultrasonic velocity and attenuation over time.

According to another aspect of the invention, there is provided a method of determining dough quality comprising:

(a) inserting a quantity of dough into a receptacle having a given thickness, said receptacle being in a chamber in which pressure can be varied;

(b) propagating an ultrasound signal through the dough at a first pressure;

(c) determining ultrasonic velocity and attenuation of the ultrasound signal after passing through the dough at said first pressure;

(d) propagating an ultrasound signal through the dough at a second pressure;

(e) determining ultrasonic velocity and attenuation of the ultrasound signal after passing through the dough at said second pressure; and

(f) predicting dough quality based on the change in ultrasonic velocity and attenuation versus pressure.

An example of a suitable range of is for example but by no means limited to zero to 2.5 bar.

In yet another embodiment of the invention, there is provided another method of determining dough quality in which the change in volume of the dough resulting from an external pressure change is measured using digital photography to determine true strain versus stress.

In yet another embodiment of the invention, there is provided a method of determining dough quality comprising:

(a) inserting a quantity of dough into a receptacle having a given thickness, said receptacle being in a chamber in which pressure can be varied;

(b) measuring the cross sectional area of the dough at a first pressure;

(c) measuring the cross sectional area of the dough at a second pressure;

(d) determining the true strain from the change in cross sectional area of the dough while maintaining a constant thickness;

(e) determining the applied stress from the difference between the second and first pressure; and

(f) predicting dough quality based on the true strain versus stress.

The pressure may be varied over more than two values.

The invention will now be described by way of example. However, it is important to note that the invention is not limited to the example and is for illustrative purposes only. It is further of note that other suitable arrangements may also be used.

To perform the velocity and attenuation measurements, the sample was sandwiched between two piezoelectric transducers. An electromagnetic (EM) pulse was generated and transmitted to one of the transducers which transformed the EM signal to an acoustic pulse with a central frequency determined by the resonant frequency of the transducers. At the transducer/sample interface, the pulse was partially transmitted into the sample and partially reflected back into the generating transducer. The pulse that travelled through the sample was then detected by the transducer at the opposite side of the sample. The receiving transducer reconverted the acoustic pulse back into an EM pulse and the output signal was amplified and displayed on the oscilloscope. In order to measure the time taken by the acoustic pulse to travel across the sample, a separate reference signal was taken with the two transducers in direct contact. An alternative is to measure the signal through a material with well-known acoustic properties. Ideally the reference and the transmitted pulses should have identical shapes and they should differ only in their time of arrival and their amplitude.

A Portable Ultrasonic Non-destructive Digital Indicating Tester (PUNDIT 6) was used to generate a short (+ve) voltage pulse (or spike). The pulse was then sent through 50Ω BNC cables to the generating transducers. The generated ultrasonic signal travelled through the sample and was detected at the other face of the sample with a similar transducer, which converted the transmitted ultrasonic signal into an EM signal. This EM signal was then amplified at the receiver amplifier (PUNDIT 6) and displayed on a digital oscilloscope. The pulse generator may be operated at an EHT voltage of either 1,200 V or 500 V, as selected by a switch at the back of the unit, and can output pulses at a pulse repetition rate of either 10 pulses per second (pps) or 100 pps. A 3.5 V positive pulse with a rise time of 2 μs, synchronized with the main output signal, was used to trigger the oscilloscope. The receiving amplifier has a high input impedance enabling the instrument to be used with piezoelectric transducers over the frequency range 5 kHz to 1 MHz.

The transducers used in these experiments have a fundamental frequency of 54 kHz. The voltage excitation of the pulse generator causes the transducer to oscillate mechanically at its own natural frequency (54 kHz).

To set the sample thickness, we first placed a number of 1-mm glass slides on the lower transducer and then lowered the top transducer until it freely rested on top of the glass slide. After that, the stopper rings on the support rods were screwed in tightly to prevent the top plate from sliding beyond the preset sample thickness. Upon raising the top plate, the sample was then placed on top of the lower transducer, and the top transducer (which was fixed to the top plate) was lowered down. The top plate was not able to slide past the preset thickness because of the stopper rings that were screwed in place on the support rods.

The data were acquired using a computer-controlled digitizing oscilloscope (Tektronic TDS 420 A) which was set in averaging mode. The signal averaging, which consisted typically of 1000 sweeps, greatly improved the signal-to-noise ratio. The triggering of the sweeps was performed by the TB synchronization output on the pulse generator, so as to synchronize the data acquisition with each repetition of the pulse from the signal generator. The oscilloscope was capable of a maximum digitizing rate of 1 GigaSample/s and could acquire record length of up to 50000 points. A general purpose interface bus (GPIB) connection between the oscilloscope and the computer allowed direct control of the data acquisition and enabled the acquired waveforms to be transmitted directly to the hard disk of the computer for subsequent analysis.

The velocity and the amplitude were calculated from the waveforms that were stored in the computer, using computer software called Microcal Origin (Microcal Software Inc.). The amplitude of each waveform was directly measured from the height of the second oscillation in volts. The reason for using the second oscillation rather than the peak of the waveform was to avoid interference effects that arise from the ringing of the transducer and possible scattering effects. The velocity on the other hand was measured by calculating the time taken for the signal to travel from one side of the sample to the other. This was done in two steps. In the first step a reference waveform was acquired. This waveform was taken with the two transducers separated by a material of well-known acoustic properties. In all of the dough experiments, the material used was an acrylic plate of thickness, d_{ref}=2 cm. The two transducers were bonded to the plates or each other via a thin coupling layer (Ultrasonic Gel II, Diagnostic Sonar Ltd.). After the reference signal was acquired, the sample was placed between the two transducers as described above, and the sample waveform was measured and downloaded to the computer. The transit time difference, Δt between the two waveforms (reference and sample) was then measured after aligning the two waveforms using the pulse shape as a guide. This time difference was then corrected for the propagation time through the acrylic reference medium (t_{ref}=d_{ref/v}_{ref}, where v_{ref} is the known ultrasonic velocity of the acrylic reference material) to give the time taken for the signal to travel through the sample, t=Δt+t_{ref}. The transit time t in conjunction with the sample thickness d yields the velocity of sound through the sample, v=d/t.

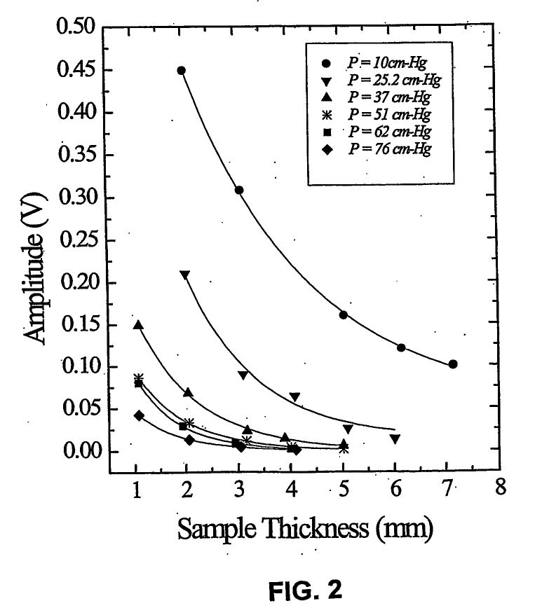

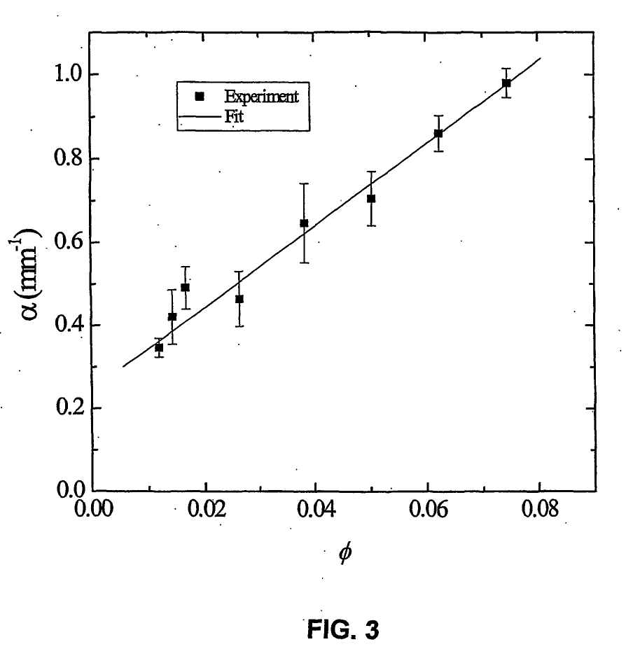

The attenuation coefficient, α, was found by measuring the amplitude of the transmitted ultrasonic signal for samples mixed at the same pressure but with different sample thicknesses ranging from 1 to 9 mm. The signal amplitudes were then plotted as a function of sample thickness and fitted by a single exponentially decay curve plus a constant background. These results are shown by the solid symbols and curves in FIG. 2, which indicates that the amplitude of the ultrasonic signal decays exponentially, as discussed above. This procedure allows spurious effects, such as bond losses and interfacial reflections, which reduce the amplitude but are independent of sample thickness, to be eliminated from the attenuation measurements, allowing the true sample attenuation to be accurately measured. This procedure was repeated for all mixing pressures and exponential decay behavior was observed for all mixing pressures, as shown in FIG. 2. These exponentially decaying curves indicate that as the mixing pressure decreases, the amplitude increases and the decay rate decreases. Therefore, as the mixing pressure is lowered, the dough becomes less absorbent and/or that there is less scattering of the ultrasonic signal. To examine this further, the attenuation coefficient, α, which was determined directly from the decay exponent of these exponential fits was plotted against the void fraction, Φ. The latter was calculated from the different mixing pressure values using equation Φ=1.113×10⁻³P, where P is the pressure. The results are plotted in FIG. 3, which shows that the attenuation coefficient increases in a linear fashion as the void fraction increases. A linear fit to the data gives,
α=0.245+9.8Φ
It is clear that the attenuation increases in proportion to the amount of air trapped in the dough, and that the air cells (or voids) make a significant contribution to a for Φ>0. The y-intercept of the above linear fit gives the background effect to the total attenuation coefficient, i.e., the contribution of the matrix to the attenuation coefficient. The total attenuation coefficient α will be a function of the amount of air introduced into the sample, Φ, during the mixing stage.

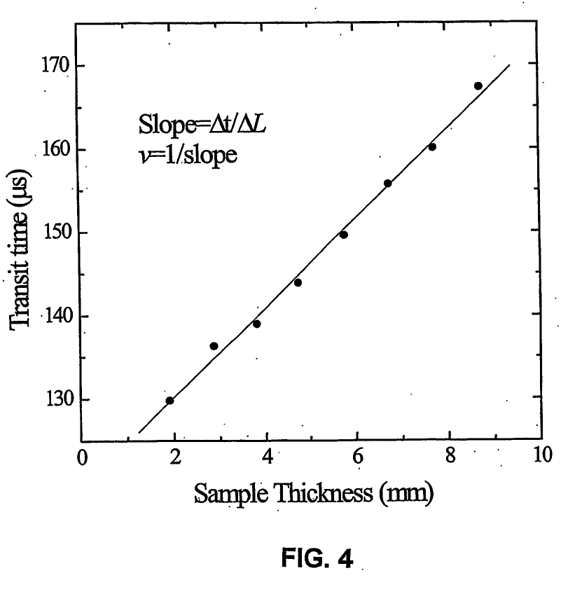

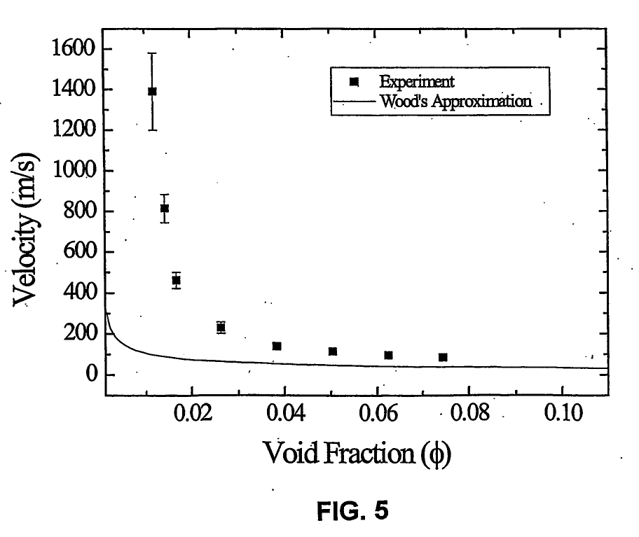

To determine the ultrasonic velocity of the dough mixed at reduced pressures, small samples of thicknesses ranging from 1 mm to 9 mm were cut from the same dough mixed at a certain pressure. By performing measurements on samples of different thicknesses, more reliable velocity and attenuation data can be acquired. For each of these small samples, we first measure the time taken for the ultrasonic signal to travel through the sample. After that, the transit time is plotted versus the sample thickness; a typical example is shown in FIG. 4. The velocity is then simply the inverse of the slope of the straight line, i.e., v=1/slope=Δd/Δt, where d is the sample thickness. Repeating this procedure for all pressures, we get the velocity dependence on mixer headspace pressure. The relationship between the velocity and the void fraction is shown in FIG. 5. The void fraction was found from mixing pressure using the above-listed equation. These data show that the velocity decreases dramatically in the range of 0.012<Φ<0.03, dropping from a velocity near to that of water to values well below the velocity of sound in air. At higher Φ values, the velocity decrease is less rapid. In the low void fraction region, the void fraction is changed by only 0.018 whereas the velocity changed by a factor of 15 from its value at Φ=0.03. The more rapid increase at these low void fractions suggests that there are mechanisms other than the void fraction which contribute to the velocity.

The addition of yeast to the dough leads to the inflation of the air bubbles. This is a direct result of the yeast fermenting the sugars to produce carbon dioxide, which diffuses through the dough matrix into the air nuclei that were occluded into the dough during the mixing stage. As a result, the void fraction, Φ, increases and the density of the dough decreases. Ultrasonic experiments done on the dough as a function of fermentation time permit us to focus on the effects of the expansion of the gas bubbles on the ultrasonic velocity and attenuation coefficient, thus providing information on the changes in the structure of the material.

The ultrasonic velocity for the fermenting dough was measured by placing the sample between two 3-cm-thick acrylic plates. Using a set of 1-mm-thick glass slides, the sample thickness was set to a pre-determined thickness. On the outer sides of the acrylic plates were the two 54 kHz transducers. Since both temperature and relative humidity are important factors in the fermentation process, the sample was placed in the proofing cabinet (37° C. and 83% R.H, as required by the baking methods, Preston et al., 1982).

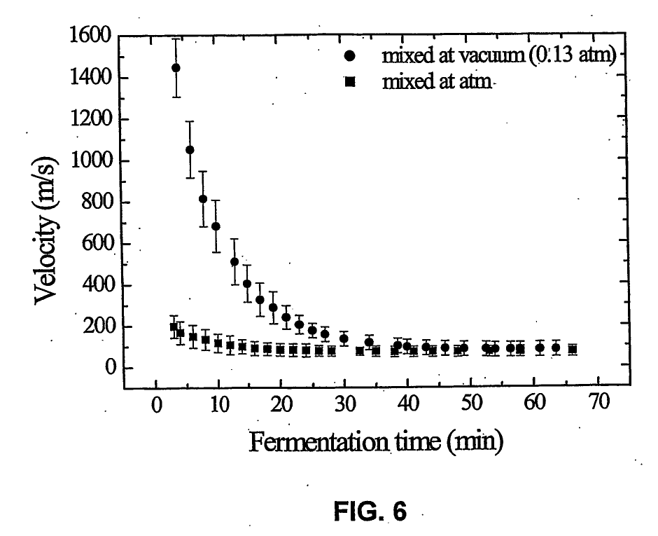

The ultrasonic velocity of the yeasted dough was found by measuring the transit time as a function of fermentation time while keeping the sample thickness fixed throughout the measurements. This velocity was then determined using v=d/t, where d is the fixed sample thickness and t is the transit time. At the beginning of fermentation, these data show that the velocity is substantially greater in the dough mixed under vacuum than its ambient counterpart (FIG. 6). This is attributed to the presence of fewer air nuclei in the dough as well as the large increase in the matrix shear modulus that was found to occur when dough is mixed at low pressures. As the yeast's metabolic activities produce CO₂ in the dough, a substantial drop in the velocity was observed in the first twenty minutes for doughs mixed under both conditions. At later fermentation times, the velocities converge to approximately the same velocity indicating that the same amount of CO₂ was produced in both doughs and that the matrix moduli are similar. Attempts to correlate the behavior of the velocity to the density decrease in the dough showed that there was no correlation between the initial drop in the velocity and the density, especially for the vacuum-mixed dough at early times where the density did not change by much. This led us to the conclusion that there are additional effects, other than the increase in void fraction, which cause this dramatic drop in velocity.

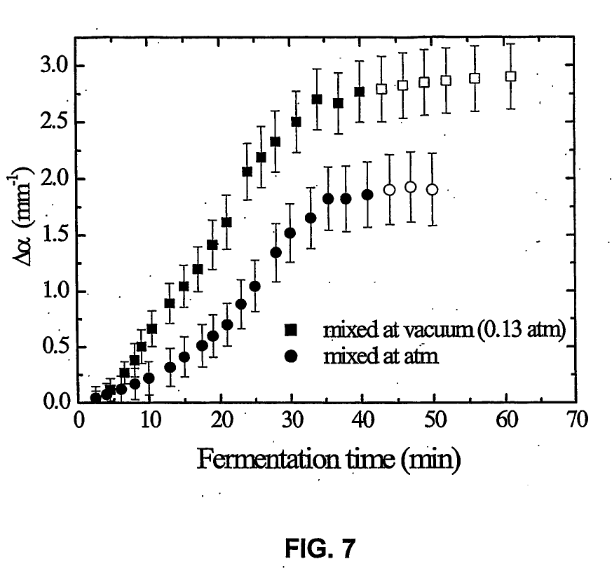

In measurements performed on unyeasted doughs (described above), the absolute attenuation coefficient, α, was determined by measuring the signal amplitude as a function of sample thickness and fitting the result to an exponential decay. The attenuation coefficient was then calculated directly from the decay length. This method requires measuring the signal amplitude for samples taken from the same dough as a function of sample thickness. For the yeasted dough this procedure was not possible because the fermenting samples evolved too rapidly for ultrasonic measurements to be made on different thicknesses of the same sample. An alternative approach is to determine the relative attenuation rather than the absolute one, i.e., measure the change in the attenuation coefficient, Δα, relative to the attenuation coefficient at the onset of fermentation. The relative attenuation Δα still provides information that is valuable for understanding the changes in the structure of the dough as a function of fermentation. The expression for Δα can be derived from the amplitude decay equation, i.e. A(t)=A₀ exp (−αL/2), where A(t) and A₀ are the signal amplitudes at times t and t=0 respectively, L is the sample thickness and α is the attenuation coefficient. Taking the signals at t=0 and at a later time t we get,
A(t)=A₀exp(−α₀L/2), at t=0
and
A(t)=A₀exp[−(α₀+Δα)L/2], at t=t₁
so that
A(t)/A(0)=exp(−ΔαL/2)
Hence,
Δα=−2/L ln [A(t)/A(0)]
Using this equation, Δα may be calculated from direct measurements of the amplitude as a function of fermentation time with A(0) as the signal amplitude at the onset of fermentation. FIG. 7 shows the relative attenuation coefficient as a function of fermentation time for the two extreme pressure values, ambient and vacuum (0.13 atm). FIG. 7 shows that for the dough mixed under vacuum, the data show a linear increase in the change of the attenuation coefficient for times between 5 and 30 minutes. After that, the signal amplitude becomes very small and the signal-to-noise ratio approaches one. Similar behavior can be seen by the data mixed at ambient pressure, with the exception that the increase in Δα is not linear at early fermentation times.

In the ultrasonic alveograph embodiments of this invention, a dough sample (made without yeast) is placed in a chamber in which a vacuum can be drawn. The transit time and amplitude of an ultrasonic pulse propagating through the dough is measured continuously (or at specific time intervals) as a vacuum is drawn to reduce pressure in the chamber to a specific end-value of pressure.

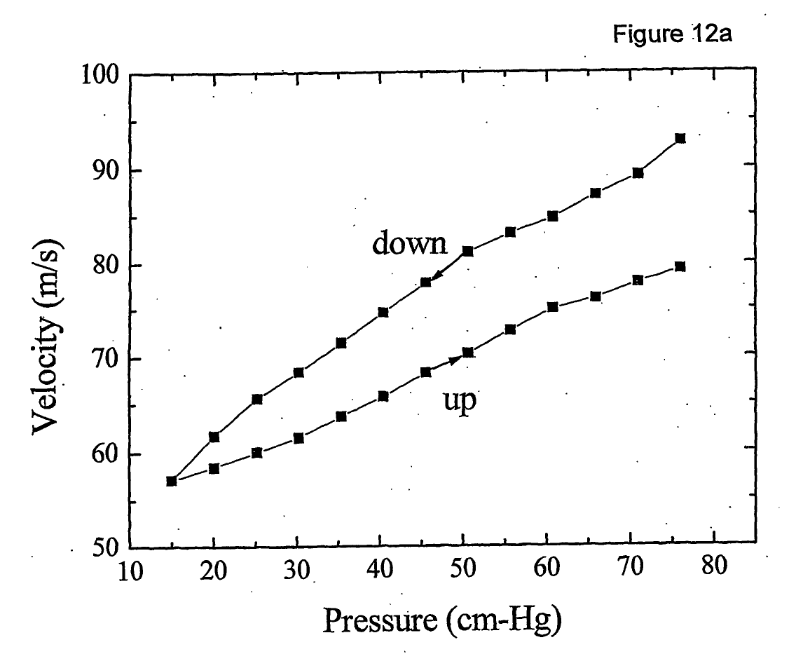

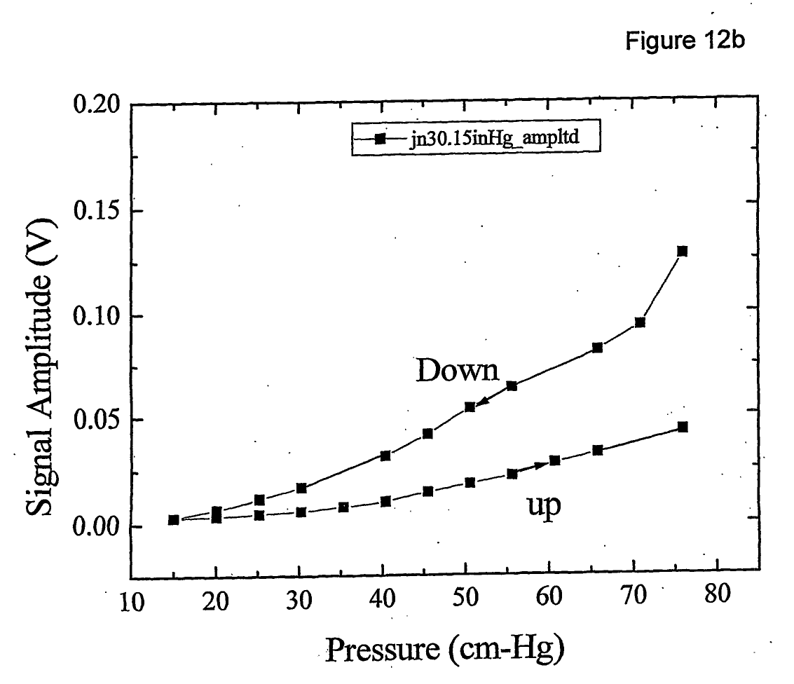

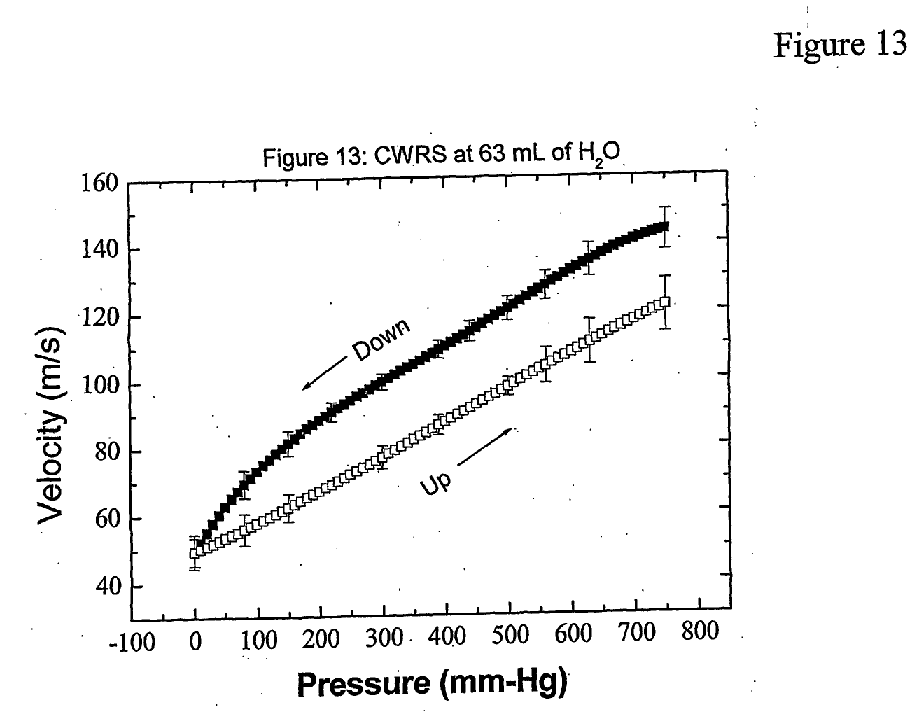

Therefore, a record of transit time and amplitude of the ultrasound signal is obtained at one thickness as a function of gas cell expansion and reduction (FIG. 12). This information can provide information on dough quality by analysing the values of transit time and amplitude at one or more pressure points to determine the corresponding changes in velocity and amplitude. The slope of transit time (or velocity) versus pressure and/or amplitude (or attenuation) versus pressure may also be used, both during expansion and reduction. The hysteresis occurring between the velocity on the decreasing and increasing pressure sweeps may also be a means of obtaining wheat quality information (FIG. 13). The latter may also be true for the hysteresis of the amplitude.

Dough relaxation following a rapid (step function) change in pressure between an initial and final pressure can also be measured by continuously monitoring the transit time and amplitude, starting just before the pressure is changed. As the gas cells expand or shrink due to the external pressure change, stress is applied to the dough matrix, and how quickly the mechanical properties of the dough respond to these stresses may be used as another indicator of dough quality. Comparing the rate of change of the ultrasonic velocity and attenuation with the rate of change in density (see below) as a result of a quasi-instantaneous pressure change will allow the changes to the dough matrix and bubble sizes to be independently measured and distinguished.

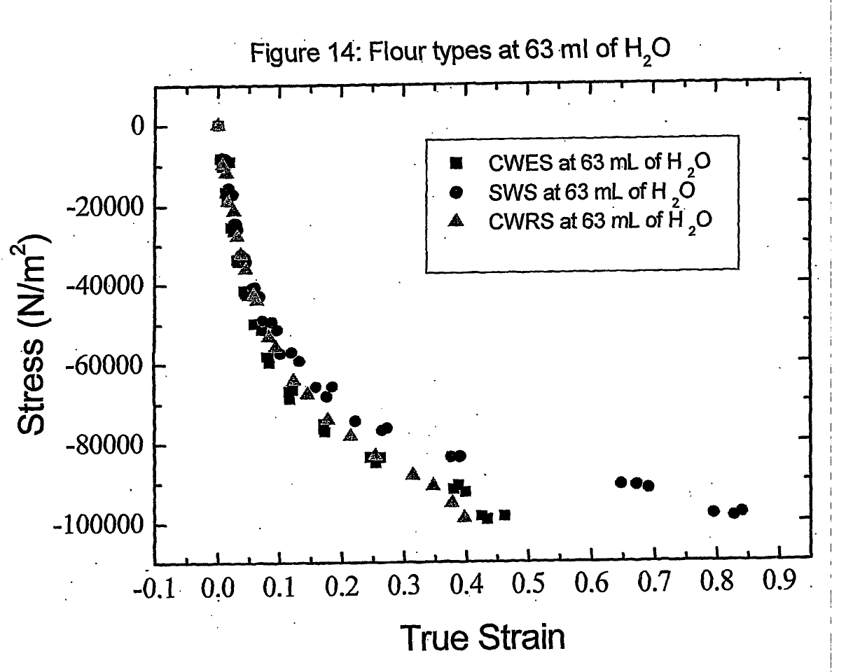

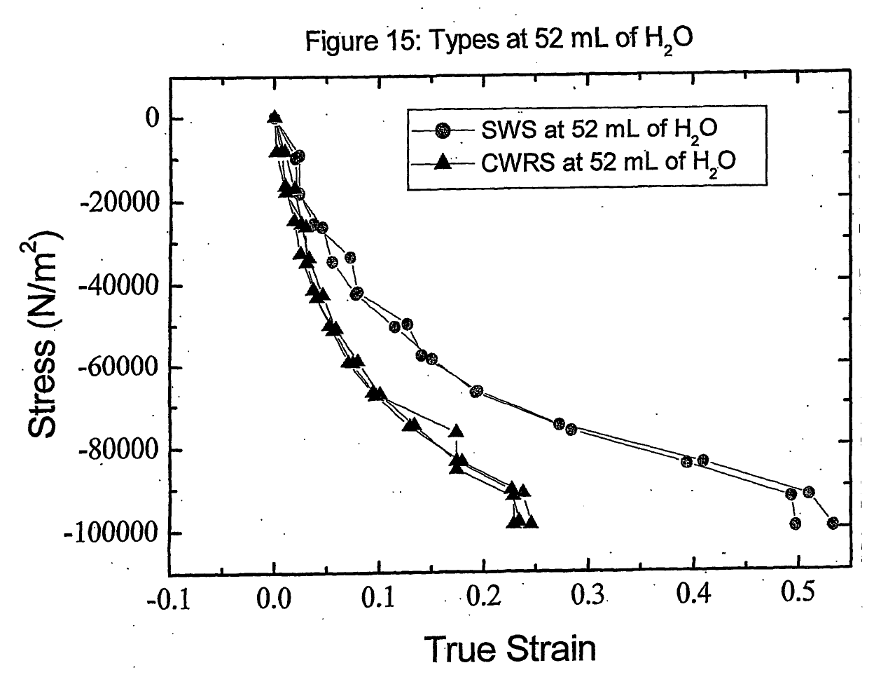

In the separate embodiment measuring the change in volume V of the dough resulting from the change in external pressure the dough sample is placed between two acrylic plates and the size of the dough slab is recorded using digital photography. The cross sectional area A(t) of the dough is then determined directly from the photographs as a function of time t as the pressure is varied using standard imaging software (e.g. Scion Image, www.scioncorn.com). By setting the thickness of the dough sample at a fixed value L, which can be made the same as for the ultrasonic measurements, and measuring the mass m of the dough sample before inserting it between the plates, the density can also easily be found from ρ=m/V(t)=m/LA(t). The method therefore allows fundamental information on the bulk expansion of the dough to be obtained as the external pressure is reduced or increased. As a result, the strain in the dough (,) can be directly measured as a function of the stress, giving another parameter that can be used to monitor dough quality. True strain is used (rather than simple or engineering strain) because of the large strains generated in the dough by the technique. In this case, each incremental increase in the size of the dough sample is expressed as a fraction of the size of the dough sample just prior to the incremental increase in size. Expressed as a function of the original size of the dough piece, the true strain is: ,=ln[A(t)/A₀] where A₀ is the initial area of the dough sample prior to application of the vacuum. Excellent reproducibility of the method has been demonstrated (FIG. 14). Since the stress in the dough is applied locally and in three dimensions, the method gives new information that complements conventional rheological measurements, in which the stress is applied at the surface of the sample. Thus, fundamental new information on dough rheology can be obtained that is potentially more relevant to assessing baking quality (FIG. 15). A second application of the method is to allow the rate of change of density or volume to be compared with the ultrasonic velocity and attenuation in the dough relaxation measurements described in the previous paragraph.

While the preferred embodiments of the invention have been described above, it will be recognized and understood that various modifications may be made therein, and the appended claims are intended to cover all such modifications which may fall within the spirit and scope of the invention.

### REFERENCES

Bushuk, W. (1975). Wheat proteins—their properties and role in breadmaking quality of flour. In Grains and Oilseeds: Handling, Marketinq, Processing (2nd ed.) (pp. 419-442). Winnipeg: Canadian International Grains Institute.
Campbell, G. M., (1991). The Aeration of Bread Dough During Mixing Ph.D. Thesis. University of Cambridge, England.
Campbell, G. M., Rielly, C. D., Fryer, P. J, and Saad, P. A. (1998). Cereal Foods World 43:163.
Eliasson, A. and Larsson, K. (1993), Cereals in Breadmaking: A Colloidal Approach. Marcel Dekker Inc., New York, USA.
Lee, H. O., Luan, H. and Daut, D. G. (1992). J. Food Eng. 16:127.
Letang, C., Piau, M. & Verdier, C. (1996). Les Cahiers de Rheologie 15:319.
Matsumoto, H. and Nishiyama, J. (1973). Cereal Chem. 50:363.
Matsumoto, H., Nishiyama, J. and Hlynka, I. (1971). Cereal Chem. 48:669.
Moorjani, R. (1984). An Investigation into the Acoustics of Bread Doughs. Master of Science Thesis, University of Leeds, Leeds. UK.
Preston, K. R., Kilborn, R. H. and Black, H. C. (1982). Can. Inst. Food Sci. Technol. J. 15(1):29.
Tipples, K. H. (1975). Breadmaking technology. In Grains and Oilseeds: Handling, Marketing, Processing (2nd ed.) (pp. 497-540). Winnipeg: Canadian International Grains Institute.
Zghal, M. C. (1999). The relationship between bread Physical texture and its structure determined by digital image analysis. Master of Science Thesis, University of Manitoba, Canada.
Zghal, C. M., Scanlon, M. G. and Sapirstein, H. D. (1999). Cereal Chem. 76:734.

1. A method of determining dough quality comprising:
a) inserting a quantity of dough into a receptacle;
b) propagating an ultrasound signal through the dough;
c) determining ultrasonic velocity and attenuation of the ultrasound signal after passing through the dough; and
d) predicting dough quality based on the ultrasonic velocity and attenuation of the ultrasound signal.

2. The method according to claim 1 wherein the ultrasonic velocity and attenuation of the ultrasound signal are determined by measuring transit time and amplitude of the ultrasound signal after passing through the dough.

3. The method according to claim 1 wherein the dough is a flour dough.

4. The method according to claim 1 wherein the ultrasound signal is a low-level ultrasound signal.

5. The method according to claim 1 wherein the ultrasound signal is an ultrasound signal in the frequency range between 18-100 kHz.

6. The method according to claim 2 wherein the transit time and the amplitude are determined relative to a reference pulse.

7. The method according to claim 1 wherein steps (a)-(c) are repeated for a second quantity of dough differing from the first quantity of dough in at least one variable affecting gas entrainment.

8. The method according to claim 7 wherein the variable affecting gas entrainment is selected from the group consisting of mixing time and headspace pressure during mixing.

9. The method according to claim 7 wherein the variable affecting gas entrainment is selected from the group consisting of bakery improving ingredients such as oxidizing improvers, enzyme improvers and surfactants (emulsifiers).

10. A method of determining dough quality comprising:
a) inserting a quantity of dough into a receptacle;
b) propagating an ultrasound signal through the dough at a first temperature;
c) determining ultrasonic velocity and attenuation of the ultrasound signal after passing through the dough;
d) repeating steps (a) through (c) at at least one other temperature; and
e) predicting dough quality based on the ultrasonic velocity and attenuation of the ultrasound signal versus temperature.

11. The method according to claim 10 wherein the ultrasonic velocity and attenuation of the ultrasound signal are determined by measuring transit time and amplitude of the ultrasound signal after passing through the dough.

12. The method according to claim 10 wherein the dough is a flour dough.

13. The method according to claim 10 wherein the ultrasound signal is a low-level ultrasound signal.

14. The method according to claim 10 wherein the ultrasound signal is an ultrasound signal in the frequency range between 18-100 kHz.

15. A method of determining dough quality comprising:
a) inserting a first quantity of dough into a first receptacle such that said first quantity of dough has a first thickness;
b) propagating an ultrasound signal through the first dough;
c) determining transit time and amplitude of the ultrasound signal after passing through the first dough;
d) inserting a second quantity of dough into a second receptacle such that said second quantity of dough has a second thickness;
e) propagating an ultrasound signal through the second dough;
f) determining transit time and amplitude of the ultrasound signal after passing through the second dough; and
g) predicting dough quality based on the ultrasonic velocity and attenuation of the ultrasound signal from the thickness dependence of the transit time and amplitude.

16. The method according to claim 15 wherein three or more samples of different thickness are analyzed.

17. The method according to claim 15 wherein the dough is a flour dough.

18. The method according to claim 15 wherein the ultrasound signal is a low-level ultrasound signal.

19. The method according to claim 15 wherein the ultrasound signal is an ultrasound signal in the frequency range between 18-100 kHz.

20. A method of analyzing fermentation response in a quantity of dough comprising:
(a) inserting a quantity of dough into a receptacle having a given thickness;
(b) propagating an ultrasound signal through the dough at a first time;
(c) determining ultrasonic velocity and attenuation of the ultrasound signal after passing through the dough at said first time;
(d) propagating an ultrasound signal through the dough at a second time;
(e) determining ultrasonic velocity and attenuation of the ultrasound signal after passing through the dough at said second time; and
(f) determining fermentation response of the dough based on the change in ultrasonic velocity and attenuation over time.

21. The method according to claim 20 wherein the ultrasonic velocity and attenuation of the ultrasound signal are determined by measuring transit time and amplitude of the ultrasound signal after passing through the dough.

22. The method according to claim 20 wherein the dough is a flour dough.

23. The method according to claim 20 wherein the ultrasound signal is a low-level ultrasound signal.

24. The method according to claim 20 wherein the ultrasound signal is an ultrasound signal in the frequency range between 18-100 kHz.

25. A method of determining dough quality comprising:
(a) inserting a quantity of dough into a receptacle such that the dough has a given thickness, said receptacle being in a chamber in which pressure can be varied;
(b) propagating an ultrasound signal through the dough at a first pressure;
(c) determining ultrasonic velocity and attenuation of the ultrasound signal after passing through the dough at said first pressure;
(d) propagating an ultrasound signal through the dough at a second pressure;
(e) determining ultrasonic velocity and attenuation of the ultrasound signal after passing through the dough at said second pressure; and
(f) predicting dough quality based on the change in ultrasonic velocity and attenuation versus pressure.

26. The method according to claim 25 wherein the ultrasonic velocity and attenuation of the ultrasound signal are determined by measuring transit time and amplitude of the ultrasound signal after passing through the dough.

27. The method according to claim 25 wherein the dough is a flour dough.

28. The method according to claim 25 wherein the ultrasound signal is a low-level ultrasound signal.

29. The method according to claim 25 wherein the ultrasound signal is an ultrasound signal in the frequency range between 18-100 kHz.

30. The method according to claim 26 wherein the hysteresis in transit time and amplitude, or velocity and attenuation, on decreasing and increasing pressure cycles is analyzed.

31. The method according to claim 26 wherein the transit time and the amplitude are measured continuously after a rapid change in pressure, thereby determining the rate of change of dough response to a quasi-instantaneous change in pressure.

32. The method according to claim 2 wherein the transit time and amplitude of the ultrasonic signal, after passing through a given thickness of dough, are used directly to monitor dough quality.

33. A method of determining dough quality comprising:
(a) inserting a quantity of dough into a receptacle having a given thickness, said receptacle being in a chamber in which pressure can be varied;
(b) measuring the cross sectional area of the dough at a first pressure;
(c) measuring the cross sectional area of the dough at a second pressure;
(d) determining the true strain from the change in cross sectional area of the dough while maintaining a constant thickness constant;
(e) determining the applied stress from the difference between the second and first pressure; and
(f) predicting dough quality based on the true strain versus stress.

34. The method according to claim 33 wherein the pressure is varied over more than two values.
